(19) United States
(12) Patent Application Publication
(10) Pub. No.: US 2025/0230388 A1
(43) Pub. Date: Jul. 17, 2025

(54) PHOTOBIOREACTOR MONITORING OF ALGAE-TO-CO2 MASS RATIOS FOR CARBON CAPTURED BY MICROALGAE

(71) Applicant: Pacific AgriTec, LLC, Santa Maria, CA (US)

(72) Inventors: William B. Clary, Newburyport, MA (US); Ramon Elias, Santa Maria, CA (US)

(21) Appl. No.: 19/030,460

(22) Filed: Jan. 17, 2025

Related U.S. Application Data
(60) Provisional application No. 63/621,991, filed on Jan. 17, 2024.

(51) Int. Cl. C12M 1/00; C12M 1/34; C12M 1/36

(52) U.S. Cl. CPC C12M 21/02; C12M 41/34; C12M 41/36; C12M 41/48

(57) ABSTRACT

A system and method for quantifying CO₂ capture by photobioreactors using microalgal biomass are disclosed. The method involves accurately quantifying carbon dioxide capture by photobioreactors using microalgal biomasses involves determining a unique ratio relating carbon consumed to biomass produced for each microalgae strain. This ratio is then used to predict CO₂ removal from flue gases or industrial processes based on the dry mass of the corresponding biomass produced. Machine learning models are also trained on data obtained from the photobioreactors to predict CO₂ consumption rates under varying conditions, thereby enhancing the accuracy and efficiency of carbon capture technologies.

### CROSS-REFERENCE TO RELATED APPLICATIONS

The present patent application claims the priority benefit of U.S. provisional patent application No. 63/621,991 filed Jan. 17, 2024, the disclosure of which is incorporated by reference herein.

### FIELD OF THE DISCLOSURE

The present disclosure is generally related to systems and methods for monitoring and determining the amount of carbon dioxide captured from gas mixtures run through photobioreactors using photosynthesis to cultivate biomass such as microalgae and cyanobacteria.

### BACKGROUND OF THE INVENTION

The capture of carbon dioxide (CO₂) by biological growth such as trees, open marine systems, and other forms of photosynthesis-based carbon sequestration has been recognized as a valuable strategy for mitigating climate change. However, the process of tracking CO₂ capture in these systems is often plagued by uncertainty due to the complex interactions between various environmental factors, nutrient availability, and biological responses. The variables that affect CO₂ capture rates in natural or semi-natural systems are numerous and can include changes in temperature, light intensity, water chemistry, and other environmental conditions.

Additionally, the presence of competing organisms, contaminants, or pests can further complicate efforts to accurately measure carbon sequestration. These complexities make it challenging to estimate the amount of CO₂ captured by these systems with any degree of certainty.

There is therefore a need in the art for improved systems and methods that effectively and efficiently mitigate the challenges of tracking carbon capture by microalgae and other biomasses, particularly when using uncontrolled growing conditions.

### SUMMARY OF THE CLAIMED INVENTION

Embodiments of the claimed invention include systems and methods for capturing, analyzing, and quantifying CO₂ capture by photobioreactors from microalgal biomass. Carbon dioxide capture may be accurately quantified by photobioreactors processing microalgal biomasses by analyzing carbon consumed to biomass produced for a specific microalgae strain and determining a unique relationship or ratio of carbon consumed to biomass produced for each microalgae strain. This ratio may then be used to predict CO₂ removal from flue gases or industrial processes based on the dry mass of the corresponding biomass produced. Machine learning models may also be trained on data obtained from the photobioreactors to predict CO₂ consumption rates under varying conditions, thereby enhancing the accuracy and efficiency of carbon capture technologies.

### BRIEF DESCRIPTION OF THE DRAWINGS

FIG. 1 illustrates an exemplary network environment in which a system for CO₂ fed photobioreactor measurement may be implemented.

FIG. 2 illustrates a flowchart illustrating an exemplary method for dehydration related to CO₂ collection and processing, algae reactor operation, and algae harvesting.

FIG. 3 illustrates an exemplary method for measuring and calculating the composition of the algal biomass.

FIG. 4 illustrates a block diagram of an exemplary computing system that may be used to implement an embodiment of the present invention.

FIG. 5 illustrates an aspect of the subject matter in accordance with one embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

Carbon retained within biomasses that is captured through photosynthesis in microalgae and cyanobacteria cells in a properly controlled photobioreactor can be accurately measured and continuously quantified. It may start with the photobioreactor operated in a properly controlled manner. For example, the water that is used and circulated within the system should be isolated and kept free of undesirable contaminants. The inlet gas stream containing carbon dioxide must be free of biological contaminants augmented by gas sterilization when needed. The inlet gas and exhaust gas mass flow rates must be known and periodically or continuously analyzed for the respective gaseous components. The supplied nutrients are managed in the same manner. The cultivated biomass that is produced is also quantified and analyzed such as for elemental composition.

As such, an accurate material balance of any specific compound or element to be tracked may be performed, such as tracking differences between the inlet and exhaust gas CO₂ content. The present technology teaches tracking and quantifying the compounds and elements, including carbon, retained by the cultivated and produced biomass.

Specifically, there may be a unique ratio that relates to the amount of carbon consumed by the harvested and dried biomass to the quantity of biomass produced under specific conditions, for different strains of microalgae or cyanobacteria. The ratio can be in the form of a single value for a specified set of operating conditions or a function that corresponds to changing operating conditions. This ratio therefore can be used to track the amount of carbon removed by the photobioreactor from gasses such as air, the products of combusted fossil fuels, and industrial processes that generate and emit gasses containing carbon dioxide. This is done by quantifying the amount of dry biomass that is produced within the photobioreactor and multiplying it by the corresponding ratio.

The ratio is determined experimentally by subjecting a specific species biomass that uses the energy from photosynthesis to consume carbon dioxide and measuring the amount of carbon that it consumes. The carbon that is consumed is expressed as CO₂ relative to the amount of biomass that is produced thus defining a ratio. Once the ratio is known, which has been determined for various microalgae species see attached chart, it can be used to reliably quantify the amount of carbon dioxide that is captured and retained by a given biomass by calculating the CO₂ removed knowing the dry mass of the species that has been produced. Now the amount of carbon dioxide that is removed from a gaseous mixture can be reliably determined using the ratio simply by knowing the incremental amount of a specific biomass that is produced. The result is a reliable and reproducible method for tracking carbon capture by an algae reactor in a CO₂ fed photobioreactor measurement system.

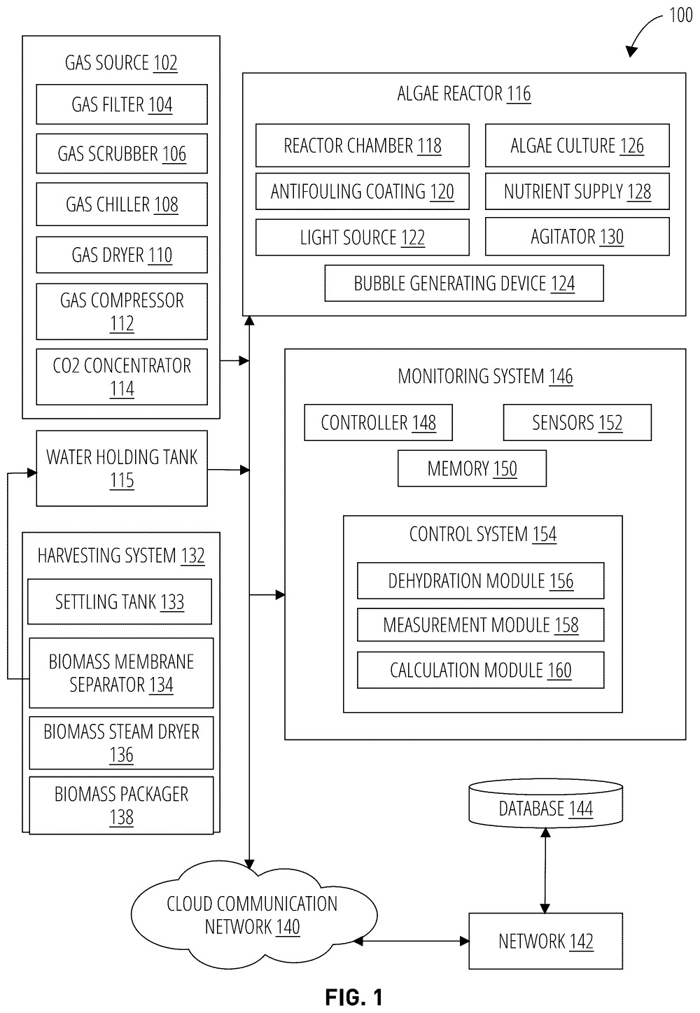

FIG. 1 illustrates a CO₂ fed photobioreactor measurement system.

The CO₂ fed photobioreactor measurement system 100 may comprise a gas source 102, which may be any source of gas, particularly those containing CO₂, such as the atmosphere or point sources. Point sources of CO₂ may include industrial processes such as combustion processes, which involve the burning of fossil fuels for energy generation, heating, or other purposes. Combustion processes in power plants, refineries, and factories release CO₂ as a result of the chemical reaction between fuel and oxygen. Concrete production is another significant point source of CO₂ emissions. Cement, a key component of concrete, is produced through a process called calcination during which limestone (calcium carbonate) is heated to high temperatures, resulting in the release of CO₂ as a byproduct. Metallurgical processes such as iron and steel production release CO₂ as a byproduct of the reduction of iron ore using carbon (coke). A gas source 102 may alternatively comprise an exhaust gas from a fermentation process, such as the fermentation of corn to create ethanol or other biofuels, or during alcohol production, etc.

The gas source 102 may include a gas filter 104 removes particulates and other contaminants from a gas stream, such as a stream of gas containing CO₂. A gas filter 104 is comprised of one or more filters designed to capture undesired particles and contaminants present in the gas. The filters used may vary depending on the nature and size of the particles to be removed. Common filter types include particulate filters, bag filters, cartridge filters, electrostatic precipitators, etc. The gas filter 104 is designed with porous media that allows the gas to pass through while trapping the particulates and contaminants. These trapped particles can include dust, ash, soot, or other solid impurities present in the gas stream. The filtration process operates based on various mechanisms, such as mechanical sieving, interception, or diffusion.

Larger particles are typically captured by mechanical sieving, as they are unable to pass through the small openings in the filter media. Smaller particles may be removed through interception, where they collide with and adhere to the filter fibers. Diffusion captures ultrafine particles by causing them to collide and stick to the filter media due to Brownian motion. Regular maintenance and periodic replacement of filters may be necessary to ensure optimal filtration efficiency. Over time, the filters may become clogged with captured particles, reducing their effectiveness. By replacing or cleaning the filters, the filtration system can continue to efficiently remove particulates and contaminants from the gas stream. Removing particulates and contaminants helps protect downstream equipment and processes from damage or fouling.

The gas source 102 may include a gas scrubber 106 typically consists of a vessel or tower filled with a scrubbing solution, such as water or a chemical reagent. The gas to be treated is directed into the scrubber, where it comes into contact with the scrubbing solution. As the gas flows through the gas scrubber 106, chemical reactions occur between the contaminants in the gas and the scrubbing solution. These reactions result in the absorption or neutralization of the contaminant chemicals or components present in the gas. The scrubbing solution acts as a medium to capture or react with the contaminants, effectively removing them from the gas. The specific design of the gas scrubber 106 can vary based on the contaminants and chemicals present in the gas. Different types of gas scrubbers 106, such as wet scrubbers or dry scrubbers, may be utilized depending on the requirements of the gas treatment process. In addition to the scrubbing solution, the gas scrubber 106 may also incorporate additional components such as filters, electrostatic precipitators, carbon absorption media, mist eliminators, or demisters. These components help remove any liquid droplets or mist formed during the scrubbing process, ensuring that the treated gas leaving the gas scrubber 106 is as free from contaminants as possible. A gas scrubber 106 helps create a suitable and safe gas stream for algae cultivation. A water holding tank 115 is a storage vessel designed to hold water received from various sources such as wells, municipal sources, water treatment facilities, or as a byproduct of industrial processes like drying a CO₂-containing gas or separating water from an algae slurry. In some cases, the CO₂ rich gas may be mixed with compressed, cooled, and de-watered air before entering the water holding tank.

The gas source 102 may include a gas chiller 108 that directs gas to a heat exchanger, which may facilitate the transfer of heat from the gas to a cooling medium. The cooled gas may then enter a gas compressor 112, which may increase its pressure for subsequent cooling and liquefaction. The gas may then be directed through a series of condensers, expansion valves, and evaporators. As the gas flows through the gas chiller 108 and exchanges heat with a refrigerant which circulates through a closed loop, the refrigerant may absorb the heat from the gas, causing the gas to cool down. The specific design and configuration of the gas chiller 108 may depend on factors such as the volume and temperature of the gas, as well as the specific requirements of the application. Different cooling techniques, such as indirect cooling using refrigerants, are employed to achieve the desired cooling and liquefaction of the gas.

The gas source 102 may include a gas dryer 110 that removes water vapor from a gas, particularly a gas containing CO₂. In some cases, a gas dryer 110 may be a desiccant dryer. This type of dryer may typically consist of a vessel filled with a desiccant material, such as silica gel or activated alumina. The gas containing CO₂ may be directed into the dryer vessel, where it comes into contact with the desiccant. The desiccant material may have a high affinity for water molecules, so as the gas passes through the dryer, the desiccant adsorbs the moisture from the gas. This adsorption process effectively removes the water vapor, leading to a reduction in the humidity of the gas. Water removed via drying processes may be recovered for use in other processes or may be sold as a product. The dried gas may then exit the gas dryer 110. To maintain the efficiency of the drying process, desiccant dryers may include regeneration heaters or blowers, which periodically regenerate the desiccant material by heating the desiccant to remove the absorbed moisture, allowing it to be reused for subsequent drying cycles. The specific design and configuration of the gas dryer 110 may vary based on factors such as the flow rate and temperature of the gas, as well as the desired moisture content. Additionally, for larger-scale industrial applications, specialized drying systems can be employed, such as adsorption dryers or membrane dryers, which offer more advanced moisture removal capabilities.

The gas source 102 may include a gas compressor 112 consists of various stages or cylinders. Each cylinder contains a piston or impeller that oscillates, reducing the volume of the gas and increasing its pressure. As the piston or impeller moves, it compresses the gas within the cylinder. This compression process involves reducing the space available for the gas to occupy, causing its molecules to come closer together increasing the density and pressure of the gas. The gas is progressively compressed as it passes through multiple stages or cylinders, with each stage contributing to a further increase in pressure. Gas compressors 112 can be driven by various sources of power, such as electric motors or engines. Depending on the application, different types of compressors may be utilized, including reciprocating compressors, rotary screw compressors, or centrifugal compressors. The specific choice of compressor depends on factors such as the desired compression ratio, flow rate, and efficiency requirements. The compressed gas containing CO₂ can be utilized for various purposes, including storage, transportation, or further processing. Higher pressure enables more efficient storage or transport of the gas, reducing the required storage or transportation volume.

In some cases, the gas source 102 may interface with or include a CO₂ concentrator 114 that increases the concentration of the CO₂. The CO₂ concentrator 114 may be a membrane or other osmotic separation device to remove oxygen, O₂, and/or nitrogen, N₂, from the gas source and thus boost the CO₂ concentration.

In some cases, a water holding tank 115 is a storage vessel designed to hold water received from various sources such as wells, municipal sources, water treatment facilities, or as a byproduct of industrial processes like drying a CO₂-containing gas or separating water from an algal slurry. In some cases, the CO₂ rich gas may be mixed with compressed, cooled, and de-watered air before entering the water holding tank

The CO₂ fed photobioreactor measurement system 100 may include a water holding tank 115 is typically constructed using materials that are resistant to corrosion and contamination, such as concrete, steel, or plastic. The primary function of a water holding tank 115 is to store water for later use or to facilitate the management and distribution of water within a system such as an algae reactor 116. The water holding tank 115 is equipped with inlet and outlet connections, allowing water to enter and exit as needed. The inlet connection is connected to one or more water sources, while the outlet connection provides a conduit for drawing water from the tank. A water holding tank 115, may include various components such as level sensors 152 or float switches to monitor and control the water level, pressure regulators to maintain consistent water pressure, and valves for controlling the flow of water in and out of the water holding tank 115.

The water holding tank 115 may operate based on the principle of maintaining a balance between water inflow and outflow. When water enters the water holding tank 115 from a source, it fills the water holding tank 115 until it reaches a predetermined level. Once the water holding tank 115 is full, the inflow is regulated, and the excess water may be redirected back to the source, to other parts of the system, or may be expelled to a drain or to the environment. In some embodiments, water may be offered for sale as a product, such as in arid climates. In such embodiments, the water may undergo further filtration and purification. An algae reactor 116, also known as a photobioreactor, is a specialized system designed to cultivate algae using a CO₂-containing gas, such as a CO₂-containing waste gas from an industrial process or combustion.

The CO₂ fed photobioreactor measurement system 100 may include an algae reactor 116, also known as an algae photobioreactor, is a specialized system designed to cultivate algae using a CO₂-containing gas, such as a CO₂-containing waste gas from an industrial process or combustion. The algae reactor comprises a reactor chamber 118 which allows light to penetrate a growth medium and supports the growth of algae. Within the reactor chamber 118, an algae slurry or suspension is maintained, which serves as the growth medium for the algae. The CO₂ containing waste gas may be fed through a bubble generating device 124 which evenly disperses the CO₂ containing gas into the algae slurry, ensuring efficient contact between the CO₂ and the algae cells. This CO₂ containing gas serves as the carbon source for algae photosynthesis.

The algae reactor 116 may be equipped with one or more light sources 122, such as LED panels, strips, tubes, or fluorescent lamps, to provide the necessary illumination for photosynthesis. The intensity and duration of light exposure may be controlled to optimize algae growth and productivity. The algae reactor 116 may incorporate a nutrient supply 128 and/or delivery system to deliver solutions containing essential elements like nitrogen, phosphorus, and potassium which may be added to the algae slurry to support algae growth The nutrient concentrations may be monitored and adjusted to maintain an optimal balance for algae cultivation. The algae reactor 116 may include a temperature regulation system to maintain the desired temperature range for optimal algae growth. This may be achieved through heating or cooling mechanisms, such as heat exchangers or temperature controllers.

The algae reactor 116 may include a reactor chamber 118 that provides a controlled environment for the cultivation of algae. The reactor chamber may be a sealed container or vessel and may be made of transparent material, such as glass or plastic, which may allow light to penetrate into the chamber. The reactor chamber 118 may contain mixing mechanisms or agitator 130, such as stirrers or circulation pumps, to ensure even distribution of the algae and nutrients throughout the reactor chamber 118.

The reactor chamber 118 may incorporate a light source 122 to provide the necessary illumination for photosynthesis. Light-emitting diodes (LEDs) or fluorescent lamps are commonly used to deliver the specific wavelengths and intensities of light required for optimal algae growth. The light source is positioned in a way that evenly distributes light across the algae slurry, ensuring uniform exposure for all algae cells. The light source may be located within the reactor chamber 118.

The algae reactor 116 may include an antifouling coating 120 that may prevent the attachment and growth of unwanted organisms, such as biofilms, algae, or other fouling agents, on the surfaces of the algae reactor 116 components. The antifouling coating 120 may comprise a specialized material or chemical formulation that inhibits the adhesion and colonization of fouling organisms. The antifouling coating 120 may be applied to the surfaces of the reactor chamber 118, including the transparent vessel, gas delivery system, and other relevant components. In some embodiments, the surfaces contacting the algae slurry possess antifouling characteristics and may not require an antifouling coating 120. The antifouling coating 120 may comprise biocides or antimicrobial agents. These agents may be incorporated into the coating formulation and may release over time to deter the growth of microorganisms and algae. The biocides act by disrupting cellular processes or inhibiting the colonization of fouling organisms, thereby preventing their attachment and growth on the coated surfaces. The antifouling coating 120 may modify surface properties such as surface energy or roughness, to create an unfavorable environment for fouling organisms. For example, hydrophobic coatings can reduce the ability of waterborne organisms to adhere to the surface, making it more difficult for them to establish and grow.

The algae reactor 116 may include a light source 122 provides the necessary illumination for photosynthesis, supporting the growth and productivity of the algae. The light source 122 in an algae reactor may emit specific wavelengths of light that are optimal for photosynthetic activity. LED panels or fluorescent lamps are commonly used as light sources due to their energy efficiency and controllable light spectrum. These light sources 122 may be positioned around and/or within the reactor to ensure uniform distribution of light throughout the algae slurry. The light source 122 operates based on the principle of converting electrical energy into light energy.

In the case of LEDs, electric current passes through a semiconductor material, causing the release of photons that generate light. Fluorescent lamps work by passing an electric current through a gas-filled tube, which results in the excitation of mercury atoms and the subsequent emission of ultraviolet light. This ultraviolet light is then converted into visible light through a phosphor coating on the inner surface of the lamp. The intensity and duration of light exposure in the algae reactor may be controlled to optimize algae growth and productivity. Light intensity may be adjusted by controlling the power supply or dimming capabilities of the light source. Additionally, timers or programmable controllers may be used to regulate the duration and timing of light exposure, simulating day and night cycles to maintain proper growth conditions for the algae or in an alternate pattern and/or frequency to optimize algae growth.

The algae reactor 116 may include a bubble generating device 124 that facilitates efficient gas-liquid contact between the CO₂-containing waste gas and the algae slurry by dispersing bubbles of the gas within the algae culture 126 to enhance the transfer of CO₂ to the algae cells. The bubble generating device 124 typically comprises of a micro-bubbler, diffuser, or membrane, which produces fine bubbles.

A CO₂ containing gas may flow through the micro-bubbler encountering small pores or perforations in the material, causing the gas to be released in the form of numerous small bubbles. These bubbles rise to the surface of the algae slurry, creating a gas-liquid interface where the CO₂ can be efficiently absorbed by the algae cells. In some embodiments, at least some of the CO₂ is solubilized into the water of the algae slurry. The rising action of the bubbles may additionally perform the function of agitating the algae. The bubble generating device 124 may include a distribution system to ensure even dispersion of the bubbles throughout the algae slurry.

The algae reactor 116 may include an algae culture 126 that is a population of algae cells which are prepared for growth within an algae reactor 116. The algae culture 126 is typically maintained as an algae slurry or suspension. The slurry contains water, nutrients, and the algae cells. The water serves as the medium for supporting algae growth, providing the necessary environment for their survival and reproduction. The algae culture 126 may be processed to remove microorganisms, such as via use of UV light, and other harmful elements, which may inhibit algae growth or compromise the efficiency of algae growth in an algae reactor 116.

The algae reactor 116 may include a nutrient supply 128 provides the necessary elements for the growth and metabolic processes of the algae culture 126. The nutrient supply 128 may comprise a controlled delivery of essential nutrients to the algae slurry which may include nitrogen (N), phosphorus (P), potassium (K), and other trace elements. The nutrient supply 128 may consist of nutrient solutions and/or additives that contain predetermined concentrations of nutrients. The nutrient supply 128 may include reservoirs or tanks that hold the nutrient solutions, dosing pumps and/or injectors that deliver the nutrients into the algae slurry, and monitoring devices to ensure control of nutrient concentrations. The dosing pumps and/or injectors may be programmed to deliver the nutrients at specific intervals or according to a predetermined schedule. The nutrient supply 128 system may additionally incorporate sensors 152 to monitor and adjust nutrient levels.

The algae reactor 116 may include an agitator 130 mixes and circulates the algae slurry, facilitating an even distribution of algae throughout the growth medium. The agitator 130 may comprise a motor or drive unit, which provides the necessary rotational force for the agitator. The motor may be connected directly, or indirectly via a shaft to one or more impellers or blades. The impellers or blades create turbulence and induce fluid motion within the algae reactor, ensuring uniform distribution of nutrients, gases, and light exposure to the algae culture 126. The shape and arrangement of the impellers may vary. The agitator 130 helps distribute nutrients evenly, ensuring uniform growth and preventing nutrient depletion or buildup in certain areas of the reactor. In some embodiments, the agitator 130 may not comprise a physical mechanism, but instead the effect may be achieved via the rising action of bubbles of CO₂ containing gas through the algae slurry.

The CO₂ fed photobioreactor measurement system 100 may include a harvesting system 132 that separates the algal biomass from the liquid medium and may utilize settling zones or baffles to increase the concentration of the algae cells. The algae slurry may be allowed to rest or undergo gentle agitation, which promotes the settling of algae cells to the bottom of the vessel. As the algae cells settle, a concentrated layer of biomass forms, while the liquid phase (supernatant) containing water and other components is decanted or drained off.

The harvesting system 132 may include a settling tank 133 that allows the algae to settle to the bottom of the tank. The denser settled algae can then be extracted for further dewatering, while the clarified water above the settled algae can be recycled to the water holding tank 114.

The concentrated algal biomass may be further dewatered using techniques such as filtration, centrifugation, or flocculation. Filtration involves passing the algae slurry through a filter medium that retains the algae cells while allowing the liquid to pass through. Centrifugation utilizes centrifugal force to separate the denser algae cells from the liquid phase. Flocculation involves the addition of chemical agents to induce the clumping of algae cells, facilitating their precipitation and separation from the liquid. In an ideal embodiment, the algae slurry is removed from the algae reactor 116 and most of the water is removed via the use of a biomass membrane separator 134. The water content of the algae may further be reduced via the use of a biomass steam dryer 136.

The harvesting system 132 may include a biomass membrane separator 134 that comprises a semi-permeable membrane having specific pore sizes that allow for the passage of liquid, dissolved substances, and smaller molecules while retaining the larger algae cells and biomass. The liquid phase, which contains water and smaller dissolved molecules, passes through the membrane and the water may be recovered and stored in a water holding tank 115. Recovered water may be reused in other processes such as in the algae reactor 116 or may be sold as a product. Via a combination of size exclusion and molecular sieving the algae cells, which are larger in size, are unable to pass through the small pores or openings of the membrane, while allowing the liquid components can permeate through the membrane structure due to their smaller size. The biomass membrane separator 134 may incorporate additional mechanisms such as backwashing or crossflow filtration. Backwashing involves periodically reversing the flow direction across the membrane surface to dislodge and remove accumulated particles and debris. Crossflow filtration utilizes a tangential flow of liquid across the membrane surface, preventing the buildup of fouling materials and maintaining optimal filtration performance. In some embodiments, a centrifuge may be used alternatively or in addition to a biomass membrane separator to remove water. Water produced by the membrane separator can be recycled by transfer to the water holding tank 115.

The harvesting system 132 may include a biomass steam dryer 136 that comprises a drying chamber or vessel, a steam generator, and a heat exchanger. The harvested algae biomass is introduced into the drying chamber, which is designed to facilitate the drying process. The steam generator generates high-pressure steam, which raises the temperature within the drying chamber, facilitating the evaporation of moisture from the algae biomass. As the algae biomass is exposed to the heated environment, the moisture within the cells begins to vaporize and then condenses on the cooler surfaces within the drying chamber, such as condensation plates or heat exchange coils before being collected and removed from the system. The drying process continues until the desired moisture content is achieved, resulting in a dry and concentrated algae biomass product. The dried biomass can then be further processed or utilized for various applications.

The harvesting system 132 may include a biomass packager 138 comprises a packaging machine or system, which may be automated or semi-automated and may include a conveyor belt, feeding mechanism, weighing system, and sealing apparatus The harvested algae biomass is fed into the biomass packager 138 by a conveyor belt or feeding mechanism. The algae is weighed to determine the desired packaging weight or quantity and is then deposited into the packaging material, such as bags or containers. The biomass packager 138 seals the package using any of heat sealing, adhesive sealing, or other mechanisms to ensure proper closure and preservation of the algae biomass. The packaging may comprise any of bulk packaging, individual bags, or containers of varying sizes.

The CO₂ fed photobioreactor measurement system 100 may a cloud communication network 140 that is comprised of a network of remote servers that are interconnected and provide various services and resources over the internet. The cloud communication network 140 may include network infrastructure, servers, storage systems, and software applications. The network infrastructure allows for seamless connectivity and communication between the algae reactor and the cloud communication network 140 servers. The servers host the necessary software applications and algorithms required for data processing, analysis, and control. The storage systems provide secure and scalable storage capacity for data generated by the algae reactor. The cloud communication network 140 may facilitate remote control and management of an algae reactor. Operators may access a cloud-based control panel or dashboard to monitor and adjust various parameters of the reactor, such as CO₂ flow rate, nutrient, and lighting conditions. The cloud communication network 140 also enables the storage and retrieval of historical data, analysis, and future optimization of the algae reactor's 116 performance.

The CO₂ fed photobioreactor measurement system 100 may include a network 142 that may be an external or internal network or platform that is utilized to enable functionality and capabilities of the algae reactor 116. The network 142 may offer various services, data analysis tools, and/or additional data or resources to optimize the algae cultivation process and improve CO₂ utilization. A network 142 may include a cloud-based platform, data analytics tools, and external APIs (Application Programming Interfaces).

Data analytics tools within the network 142 may enable advanced data processing and insights generation, for example by utilizing machine-learning algorithms that can identify patterns in growth rates, photosynthetic efficiencies, and other key metrics to predict and prevent potential issues. Additionally, the network 142 may incorporate IoT (Internet of Things) connectivity to allow real-time monitoring and control of the photobioreactor system, enabling remote access and management by authorized personnel. Furthermore, the network may integrate with other systems and devices, such as sensors for CO₂ levels, temperature, pH, and dissolved oxygen, to provide a comprehensive view of the algae cultivation process and enable data-driven decision making. By leveraging these advanced technologies, the network 142 can help optimize the performance of the photobioreactor system and improve overall efficiency in CO₂ utilization.

These tools may further employ algorithms and machine-learning techniques to analyze the data collected from the algae reactor to provide valuable insights into such as CO₂ consumption rates, optimal nutrient dosage, growth patterns, and other performance indicators. These insights can be used to optimize the operation of the algae reactor 116, improve CO₂ utilization efficiency, and improve overall productivity. Furthermore, the network 142 may offer external APIs that allow seamless integration between the algae reactor and external systems or services. These APIs enable data exchange and interoperability, facilitating collaborations with environmental monitoring agencies and/or industry partners including validation of carbon collected and stored within the produced algae biomass.

The CO₂ fed photobioreactor measurement system 100 may include a database 144 that may be an internal or an external database which may store data including operational parameters, environmental conditions, growth metrics, and historical records relating to an algae reactor. The database 144 may store data related to growth characteristics of an algae culture 126 created by a vendor who prepares algae cultures 126. Similarly, a database 144 may comprise manufacturer information relating to any component of an algae reactor 116 including gas collection and biomass harvesting. In some cases, the predetermined correlation threshold may be adjusted or calibrated based on the data from the databases 144.

In some cases, to optimize the performance of the photobioreactor system and improve overall efficiency in CO₂ utilization, advanced machine learning techniques can be applied to analyze the data collected from the algae reactor. Supervised learning can be employed to train models on labeled data to predict outcomes based on input features, such as predicting CO₂ consumption rates based on environmental conditions and operational parameters, identifying optimal nutrient dosages for maximum algal growth, forecasting growth patterns, and detecting anomalies in the algae culture.

Unsupervised learning can also be used to identify patterns or structure in the data, such as clustering algae cultures based on their growth characteristics, environmental conditions, or operational parameters, and identifying outliers and anomalies that may indicate issues with the photobioreactor system. Reinforcement learning can be employed to train models to make decisions based on rewards or penalties, optimizing operational parameters for maximum CO₂ utilization and algal growth, and developing control strategies to maintain optimal conditions within the photobioreactor system.

Deep learning techniques can also be used to analyze complex data patterns, such as analyzing multi-dimensional data from sensors (e.g., temperature, pH, CO₂ levels) to predict CO₂ consumption rates and optimal operational parameters and identifying patterns in historical data to forecast future performance of the photobioreactor system. These machine learning techniques can be applied to various data sources, including sensor readings, image analysis, and historical records. Specifically, for estimating the amount of CO₂ captured by the algae, machine learning techniques such as photography and image analysis can be employed to analyze high-resolution images of the algae culture, using computer vision techniques (e.g., image segmentation, object detection) to estimate biomass density and growth rates. Sensor data fusion can also be used to fuse together sensor data from CO₂ levels, temperature, pH, and dissolved oxygen sensors, providing more accurate estimates of CO₂ consumption rates.

Algal growth modeling and CO₂ consumption modeling can also be employed to predict algal growth rates based on environmental conditions and operational parameters, and predicting CO₂ consumption rates based on environmental conditions, operational parameters, and algal growth rates. A predictive model for estimating CO₂ captured by the algae can involve collecting data from various sources, extracting relevant features from the collected data (e.g., environmental conditions, operational parameters, algal growth metrics), training a machine learning model on the feature-engineered data to predict CO₂ consumption rates, and evaluating the performance of the predictive model using metrics such as mean absolute error (MAE) or mean squared error (MSE). By employing these machine learning techniques, the CO₂ fed photobioreactor measurement system 100 can provide more accurate estimates of CO₂ captured by the algae, enabling data-driven decision making and optimization of the photobioreactor system.

The CO₂ fed photobioreactor measurement system 100 may include monitoring system 146 for the algae reactor 116 that comprises at least a controller 148, memory 150, and sensors 152 within the algae reactor 116 which measure parameters such as CO₂ concentration, temperature, pH level, dissolved oxygen, nutrient levels, etc. The sensors 152 may continuously collect data, providing real-time information about the status of the algae reactor 116. The collected data from the sensors 152 may be transmitted to a central control unit or system. The control unit processes and analyzes the data and may modify one or more parameters of an algae reactor 116 in response to the data collected from one or more sensors 152. For example, the monitoring system 146 may control the flow rate of the CO₂-containing waste gas, adjust the lighting intensity and duration, regulate the nutrient dosing, monitor the overall algae reactor 116 conditions, etc.

The control unit may further communicate with a user interface which displays sensor 152 readings and other relevant information. The monitoring and monitoring system 146 may additionally or alternatively comprise a brokerage and/or arbitration function. For example, the monitoring and monitoring system 146 may determine the amount of CO₂ captured by harvested algae biomass, and may further determine a carbon credit value and/or facilitate the sale of the algae biomass, carbon credits, process byproducts, or other products and services related to an algae reactor 116 including equipment and/or licensing, maintenance and/or operational services, etc.

The monitoring system 146 may include the controller 148 that is a computing device comprised of a processor for performing computations and communicates with a memory 150 for storing data. The controller 148 may be in communication with a communications interface and/or one or more sensors 152 and may further be allowed to control the at least one function or process related to an algae reactor 116 and/or the brokerage and/or arbitration of products and/or services related to an algae reactor 116. The controller 148 may be a commercially available central processing unit (CPU) or graphical processing unit (GPU) or may be a proprietary, purpose-built design. More than one controller 148 may operate in tandem and may be of different types, such as a CPU and a GPU. A GPU is not restricted to only processing graphics or image data and may be used for other computations.

The monitoring system 146 may include memory 150 that serves as the electronic circuitry within a computing device that temporarily stores data for usage by the controller 148. The memory 150 may additionally comprise persistent data storage for storing data used by the controller 148. The memory 150 may be integrated into a controller 148 or may be a discrete component. The memory 150 may be integrated into a circuit, such as soldered on component of a single board computer (SBC) or may a removable component such as a discrete dynamic random-access memory (DRAM) stick, secure digital (SD) card, flash drive, solid state drive (SSD), magnetic hard disk drive (SSD), etc. In some embodiments, memory 150 may be part of a controller 148. Multiple types of memory 150 may be used.

The monitoring system 146 may include sensors 152 that collect data by measuring one or more physical properties such as CO₂ concentration, temperature, pH level, dissolved oxygen, and nutrient levels, etc. CO₂ sensors 152 are essential for measuring the concentration of CO₂ in the algae reactor 116 and may utilize infrared or electrochemical technology to detect and quantify the CO₂ concentration in the gas phase by measuring the absorption or electrical properties of a gas sample and converting it into a CO₂ concentration value. Temperature sensors 152 may comprise thermocouples, resistance temperature detectors (RTDs), optical temperature sensors, etc. pH sensors 152 monitor the acidity or alkalinity of an algae slurry and may utilize pH-sensitive electrodes and/or indicators that generate electrical signals corresponding to the hydrogen ion concentration in the liquid medium. Nutrient sensors 152 may monitor the levels of essential nutrients such as nitrogen, phosphorus, and potassium within the algae reactor 116.

The control system 154 sequentially initiates a dehydration module 156 which removes the water from an algae slurry, resulting in a dried algae product, the measurement module 158, which measures multiple parameters related to the algae, such as mass, water content, and percent composition, and then the calculation module 160, which retrieves and selects one or more formulas and/or conversion factors, and determines the amount of CO₂ captured by the algae, accounting for at least water concentration and percent composition of other components of the algae. The dehydration module 156 removes water from an algae slurry resulting in a dried algae product. The algae drying process may comprise multiple steps or stages, such as a dewatering step, for mechanically removing water from the algae, and a further drying step which may utilize thermal energy to evaporate water from the algae. The dried algae product is sent to the control system 154.

The measurement module 158 receives a dried algae product and measures the mass, water concentration, and percent composition of other components and returns the measurements to the control system 154. The calculation module 160 receives measurements of algae including mass, water content, and percent composition of other components, retrieves and selects one or more formulas and/or conversion factors, and calculates the amount of CO₂ captured by the algae. The calculation may comprise two steps, first determining the amount of CO₂ captured without considering additional components in the algae, and a second step whereby the calculated amount is adjusted, such as by subtracting a calculated mass of water and/or a calculated mass of other components within the algae not comprised of carbon and oxygen. The resulting amount of carbon dioxide is sent to the control system 154.

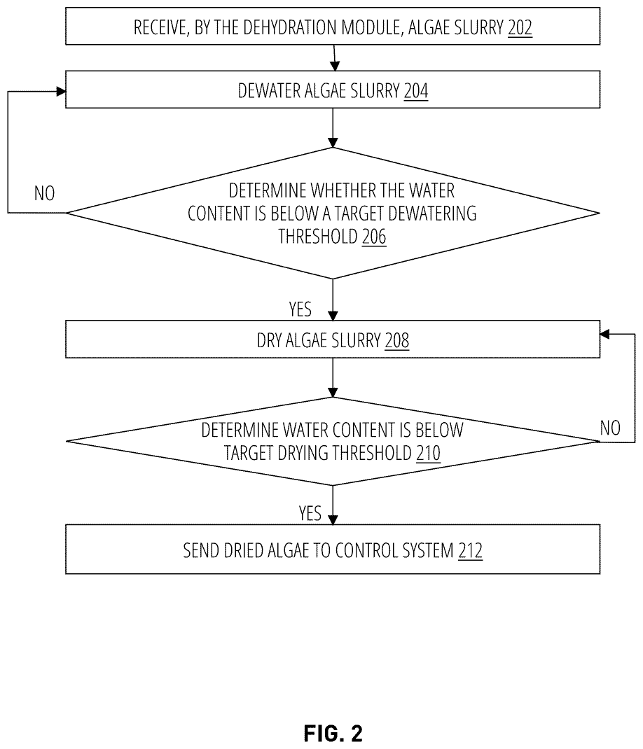

FIG. 2 illustrates a flowchart illustrating an exemplary method for dehydration related to CO₂ collection and processing, algae reactor operation, and algae harvesting. The method of FIG. 2 may be performed based on execution of a dehydration module 156 by a processor in accordance with initiation and call by control system 154.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In some cases, the control system 154 may initiate the dehydration module 156. The dehydration module 156 may receive an algae slurry from an algae reactor 116, and removes water from the algae slurry via a multistep process, such as by removing the majority of the water via a dewatering process which may utilize a biomass membrane separator 134 or a centrifuge, and further dries the algae using a biomass steam dryer 136 or other drying mechanism such as an oven, heated air, evaporation, etc. In some cases, dried algae may be received from the dehydration module 156. The dried algae may have a predetermined water content, be within a range of water content, or under a threshold.

More specifically, the exemplary method 200 begins with receiving at step 202, from an algae reactor 116. The algae slurry enters a harvesting system 132 such as via a process of drawing off part or all of the algae slurry.

At step 204, the dehydration module 156 may dewater an algae slurry such as via a biomass membrane separator 134. In some embodiments, dewatering algae may comprise the use of a centrifuge system which uses rotation to separate water from the algae cells. In an embodiment, utilizing a centrifuge to reduce the water content of the algae slurry.

At decision block 206, the dehydration module 156 may determine whether the water content is below a target dewatering threshold. In an embodiment, the dewatering threshold is 25%, and the water concentration of the algae slurry after processing in the centrifuge is 20%, therefore the water content is below the target dewatering threshold. In an alternate embodiment, the water concentration of the algae slurry is 30%, which is not below the target dewatering threshold, and therefore continuing to dewater the algae slurry. In some embodiments, the dewatering threshold may be set by a human operator. In other embodiments, the dewatering threshold may be selected by an algorithm, such as utilizing machine learning to maximize any of water recovery, energy efficiency, algae yield, etc. In some embodiments, the dewatering threshold may be determined upon the required starting water concentration of an algae slurry prior to beginning a drying process.

At step 208, the dehydration module 156 may dry the dewatered algae slurry such as via a biomass steam dryer 136. In alternate embodiments, the dewatered algae slurry may alternatively be dried using an oven, heated air, evaporation, etc. The algae slurry may further be dried using a plurality of methods.

At decision block 210, the dehydration module 156 may determine whether the water content of the dried algae slurry is below a target drying threshold. In an embodiment, the target drying threshold is 5%. In an embodiment the water concentration of the algae is 1% which is less than the 5% threshold. In an alternate embodiment, the water concentration of the algae is 10% which is less than the 5% threshold, therefore continuing to dry the dried algae slurry. The drying threshold may be set by a human operator, determined by an algorithm, such as utilizing machine learning to maximize any of water recovery, energy efficiency, algae yield, etc. In some embodiments, the drying threshold may be determined based upon the needed water concentration for a desired algae product. At step 212, the dehydration module 156 may send the dried algae to the control system 154.

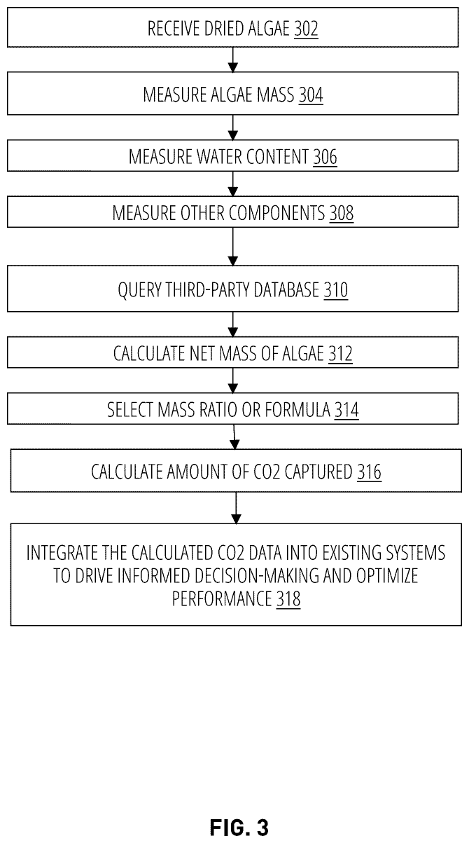

FIG. 3 illustrates an exemplary method for measuring and calculating the composition of the algal biomass. The method of FIG. 3 may be performed based on execution of the measurement module 158 and the calculation module 160 by a processor in accordance with initiation and call by the control system 154.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In some cases, the control system 154 may initiate the measurement module 158. The measurement module 158 may receive dried algal biomass and measures the mass, water content, and percent composition of other components in the algae not comprised by algal biomass, or water such as other biological components (e.g. bacteria), nitrogen, potassium, or trace elements or minerals including metals such as copper, zinc, manganese, iron, etc. The measurements of the algae may be received from the measurement module 158. The measurements may comprise mass, water content, and percent composition of other components in the algae.

In some cases, the control system 154 may initiate the calculation module 160. The calculation module 160 may receive algae measurements comprising mass, water content, and percent composition of other components in the algae and queries a database 144 for one or more formulas and/or conversion factors, from which, one or more are selected for use in calculating the amount of CO₂ captured. The amount of CO₂ captured may first be calculated without considering the impact of the water content or percent composition of other components, after which an additional calculation may be performed to account for the water content of the algae and the percent composition of other components within the algae. The control system 154 may receive the calculated amount of CO₂ from the calculation module 160.

More specifically, the exemplary method may begin with receiving at step 302, dried algal biomass from the control system 154. The dried algal biomass may comprise a final water concentration as required for an algal product or may be received at an intermediary state with higher than the final water concentration.

At step 304, the measurement module 158 may measure the mass of the algae. The mass may be measured via any method which may include measuring one or more of weight, volume, density, etc. of the dried algal biomass. In an embodiment, measuring the weight of the algae. In some embodiments, the mass may be measured using multiple methods, and an average may be utilized.

At step 306, the measurement module 158 may measure the water content of the algae. In some embodiments, a moisture and/or humidity measurement may be used. In other embodiments, the water content may be measured based upon the difference between measuring the mass of a sample of the algae before and after a process of drying the sample until its mass no longer changes. Water content may alternatively be measured via a moisture meter or sensors such as a near-infrared moisture sensor. In other embodiments, the water content may be determined using mass spectrometry. In some embodiments, the water content may be measured using multiple methods.

At step 308, the measurement module 158 may measure the mass fraction of components other than algal biomass which was consumed as carbon dioxide, and nutrients from the water including nitrogen and phosphorus during photosynthesis. Examples of such components are biological components like bacteria, nitrogen, potassium, or minerals and trace elements including metals such as copper, zinc, manganese, iron, etc. The additional components may be from the algae, or may be from residual fertilizer, pH buffer solutions, contaminants, etc. which may not have been removed during the drying process.

Measurement of other components may be executed using methods such as mass spectrometry, where a sample of the algae is analyzed for elemental composition expressed as mass fractions. Mass spectrometry may comprise analysis of the composition to determine component elements and may further extrapolate compounds. The measurements may comprise a mass of dried algal biomass, a percent water content, and a percent composition of other components in the algae. The algal biomass measurements may comprise at least a mass, weight, or volumetric measurement of algal biomass which may further comprise a density measurement and/or calculation. The measurements may additionally comprise water content and the fractional composition of other components, such as may be represented by a mass fraction.

The exemplary method may continue with step 310, the calculation module 160 querying a third-party database 144 for any of a mass fraction, conversion factor, formula, etc. representing the composition of the species of algae comprising the biomass. In some cases, the mass fraction may be represented as an empirical formula in the form CₐN_{b}O_{c}P_{d}SₑH_{f}K_{g}CaₕMgᵢFeⱼ wherein each letter a-j represents a relative fractional mass of the species of algae. In an embodiment, Chlorella Vulgaris has a mass fraction represented by the values: a=1; b=0.084; c=0.525; d=0.002; e=0.002; f=1.995; g=0; h=0; i=0; j=0. In some embodiments, the empirical formula may vary, such as including additional elements. This may be the case in yet undiscovered species of algae and/or genetically modified algae or other sources of biomass.

The table below illustrates example algae empirical formulas:

CₐN_{b}O_{c}P_{d}SₑH_{f}K_{g}CaₕMgᵢFeⱼ

| | | a | b | c | d | e | f | g | h | i | j |
|---|---|---|---|---|---|---|---|---|---|---|---|
| 1. | Aphanothece microscopica Nägeli | 1.000 | 0.184 | 0.518 | 0.040 | 0.005 | 1.999 | 0.048 | 0.014 | 0.026 | 0.0000 |
| 2. | Botryococcus braunii | 1.000 | 0.069 | 0.233 | 0.007 | 0.001 | 1.838 | 0.000 | 0.000 | 0.000 | 0.0000 |
| 3. | Chaetoceros sp. | 1.000 | 0.193 | 0.431 | 0.035 | 0.004 | 1.923 | 0.032 | 0.009 | 0.017 | 0.0000 |
| 4. | Chaetoceros cakitmns | 1.000 | 0.170 | 0.602 | 0.070 | 0.003 | 1.926 | 0.079 | 0.023 | 0.042 | 0.0001 |
| 5. | Chaetoceros calcitrans f pumilus | 1.000 | 0.170 | 0.426 | 0.027 | 0.002 | 1.777 | 0.000 | 0.000 | 0.000 | 0.0000 |
| 6. | Chaetoceros cf. wighamii | 1.000 | 0.255 | 0.478 | 0.040 | 0.003 | 1.732 | 0.000 | 0.000 | 0.000 | 0.0000 |
| 7. | Chaetoceros gracilis | 1.000 | 0.298 | 0.513 | 0.069 | 0.001 | 1.491 | 0.000 | 0.000 | 0.000 | 0.0000 |
| 8. | Chaetoceros muelkri | 1.000 | 0.217 | 0.452 | 0.032 | 0.003 | 1.777 | 0.000 | 0.000 | 0.000 | 0.0000 |
| 9. | Chlatnydomonas reinhardtii | 1.000 | 0.162 | 0.360 | 0.011 | 0.003 | 1.909 | 0.000 | 0.000 | 0.000 | 0.0000 |
| 10. | Chlamydomonas sp. | 1.000 | 0.048 | 0.544 | 0.001 | 0.001 | 1.982 | 0.000 | 0.000 | 0.000 | 0.0000 |

At step 312, the calculation module 160 may calculate the net mass of the algal biomass by subtracting the mass of the water, non-algal biomass, and other elemental and mineral weights. In some embodiments, this calculation is performed using absolute or relative measurements for each relevant component. In some cases, the calculation is performed by first removing the water component based upon the percentage water content. The amount remaining may be resolved based upon the known mass fraction of the algae species and may further utilize a control element, such as carbon, the source of which can be attributable only to the algal biomass and not other materials.

At step 314, the calculation module 160 may select a mass fraction, ratio, or formula to use to calculate the amount of CO₂ captured by the algae. In some cases, the formula may be a conversion factor, such as 1.86 for Chlorella Vulgaris, such that for each kg of algal mass, 1.86 kg of carbon dioxide would be captured. In other embodiments, multiple formulas and/or conversion factors may be selected.

At step 316, the calculation module 160 may calculate the amount of CO₂ captured by the algae. For Chlorella Vulgaris, the empirical formula is given as CₐN_{b}O_{c}P_{d}SₑH_{f}K_{g}CaₕMgᵢFeⱼ where the coefficients are a=1; b=0.084; c=0.525; d=0.002; e=0.002; f=1.995; g=0; h=0; i=0; j=0. The molecular weight of Chlorella Vulgaris is given by: 1×12.011+0.084×14.01+0.525*16+0.002×30.97+0.002×32.06+1.995×1.008=23.725. The molecular weight of CO₂ is 44.009, therefore the mass ratio of CO₂ to algal mass of Chlorella Vulgaris is 44.009/23.725=1.855.

In some cases, the ratio of a specific algae species to carbon dioxide captured may be stored in the database 144 and the ratio may be used without being independently calculated. Multiplying the algal mass by the ratio of 1.855 may yield the amount of CO₂ captured. For example, if 1500 kg of algal mass of Chlorella Vulgaris are harvested, the amount of CO₂ captured would be 2782.5 kg.

At step 318, the calculation module 160 may integrate the calculated CO₂ data into existing systems to drive informed decision-making and optimize performance.

For example, with the calculated amount, accurate monitoring and tracking of carbon capture by microalgae is enabled. This reproducible method takes into account the unique ratio specific to each microalgae strain, allowing for reliable quantification of CO₂ capture using known dry mass values. As a result, precise management of biomass production and CO₂ removal becomes possible. In some cases, the CO₂ fed photobioreactor measurement system 100 may be triggered to receive a certain mass of algal slurry based on the calculated CO₂ data for a next carbon capture process.

In some cases, by feeding CO₂ capture data into a computerized control system, precise control over system parameters such as temperature, pH levels, and nutrient supply becomes possible. This leads to improved efficiency and productivity in algae growth rates, resulting in increased biomass production and CO₂ removal capabilities. Furthermore, the optimized system settings can be stored and retrieved for future reference, allowing for seamless integration with other systems.

In some cases, the calculated CO₂ capture data is integrated with existing business intelligence systems to provide real-time insights for decision-making. This enables informed optimization of resource allocation, prediction of market trends, and strategic planning for the algae reactor system. By leveraging this data-driven approach, key performance indicators can be tracked in real-time, ensuring that decisions are made based on accurate and up-to-date information. The CO₂ capture data may be used to generate real-time analytics reports, providing stakeholders with immediate insights into system performance. This enables informed decision-making and strategic planning, ensuring that key performance indicators are tracked in real-time. By leveraging this real-time approach, operational efficiency and productivity can be continuously improved.

In some cases, using CO₂ capture data to train machine learning models allows for more accurate predictions of algae growth rates, CO₂ consumption patterns, and other relevant factors. This enables better decision-making and strategic planning for the algae reactor system, resulting in improved efficiency and productivity. By leveraging machine learning models, predictive maintenance scheduling can be further optimized, ensuring that system performance is maintained at optimal levels. Using CO₂ capture data to train machine learning models allows for precise control over system parameters such as temperature, pH levels, and nutrient supply. This enables real-time adjustments to be made to optimize operational performance, resulting in improved efficiency and productivity in algae growth rates.

Additionally, accurate monitoring and tracking of system performance may further be enabled by integrating the CO₂ capture data with predictive maintenance algorithms. This reproducible method takes into account the unique operational characteristics specific to each algae reactor model, allowing for reliable prediction of impending issues and enabling proactive maintenance scheduling. As a result, downtime is minimized, and optimal system performance is ensured.

In some cases, the calculated CO₂ capture data is stored and analyzed in a cloud-based environment, allowing for scalable storage, real-time analytics, and secure access from anywhere. This ensures that critical information is readily available to stakeholders, enabling informed decision-making and strategic planning. Furthermore, the cloud-based architecture enables seamless integration with other systems, ensuring that data is always up-to-date and accurate. Furthermore, by implementing advanced cybersecurity measures, sensitive CO₂ capture data is protected from unauthorized access or tampering, ensuring confidentiality, integrity, and availability of critical information. This ensures that stakeholders can trust the accuracy and reliability of the calculated CO₂ capture data, enabling informed decision-making and strategic planning.

The calculated CO₂ capture data may also be integrated with automatic process control systems to ensure precise control over system parameters such as temperature, pH levels, and nutrient supply. This enables real-time adjustments to be made to optimize operational performance, resulting in improved efficiency and productivity in algae growth rates. The CO₂ fed photobioreactor measurement system 100 may be further integrated with Internet of Things (IoT) devices, such as sensors, actuators, or other smart devices, to create a more comprehensive and connected ecosystem. This enables real-time monitoring and tracking of system performance, allowing for proactive maintenance scheduling and optimization of operational parameters. By integrating IoT devices, the algae reactor system becomes even more efficient and productive.

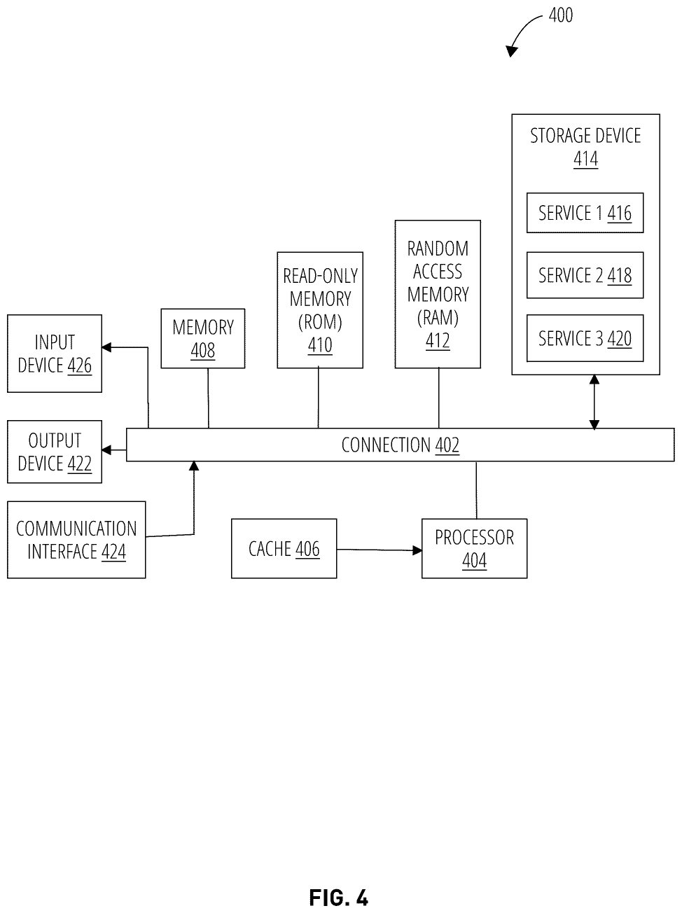

FIG. 4 illustrates a block diagram of an exemplary computing system that may be used to implement an embodiment of the present invention. The example of computer system 400 can be for example any computing device making up CO₂ fed photobioreactor measurement system 100, or any component thereof in which the components of the system are in communication with each other using connection 402. Connection 402 can be a physical connection via a bus, or a direct connection into processor 404, such as in a chipset architecture. Connection 402 can also be a virtual connection, networked connection, or logical connection.

In some embodiments, computing computer system 400 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example computing computer system 400 includes at least one processing unit (CPU or processor) 404 and connection 402 that couples various system components including system memory 408, such as read-only memory (ROM) 410 and random access memory (RAM) 412 to processor 404. Computing system 500 can include a cache of high-speed memory 408 connected directly with, in close proximity to, or integrated as part of processor 404.

Processor 404 can include any general purpose processor and a hardware service or software service, such as services 416, 418, and 420 stored in storage devices 414, configured to control processor 404 as well as a special-purpose processor where software instructions are incorporated into the actual processor design. Processor 404 may essentially be a completely self-contained computing system, containing multiple cores or processors, a bus, memory controller, cache, etc. A multi-core processor may be symmetric or asymmetric.

To enable user interaction, computing computer system 400 includes an input device 426, which can represent any number of input mechanisms, such as a microphone for speech, a touch-sensitive screen for gesture or graphical input, keyboard, mouse, motion input, speech, etc. Computing system 400 can also include output device 422, which can be one or more of a number of output mechanisms known to those of skill in the art. In some instances, multimodal systems can enable a user to provide multiple types of input/output to communicate with computer system 400. Computing system 400 can include communication interface 424, which can generally govern and manage the user input and system output. There is no restriction on operating on any particular hardware arrangement, and therefore the basic features here may easily be substituted for improved hardware or firmware arrangements as they are developed.

Storage device 414 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 414 can include software services, servers, services, etc., that when the code that defines such software is executed by the processor 404, it causes the system to perform a function. In some embodiments, a hardware service that performs a particular function can include the software component stored in a computer-readable medium in connection with the hardware components, such as processor 404, connection 402, output device 422, etc., to carry out the function.

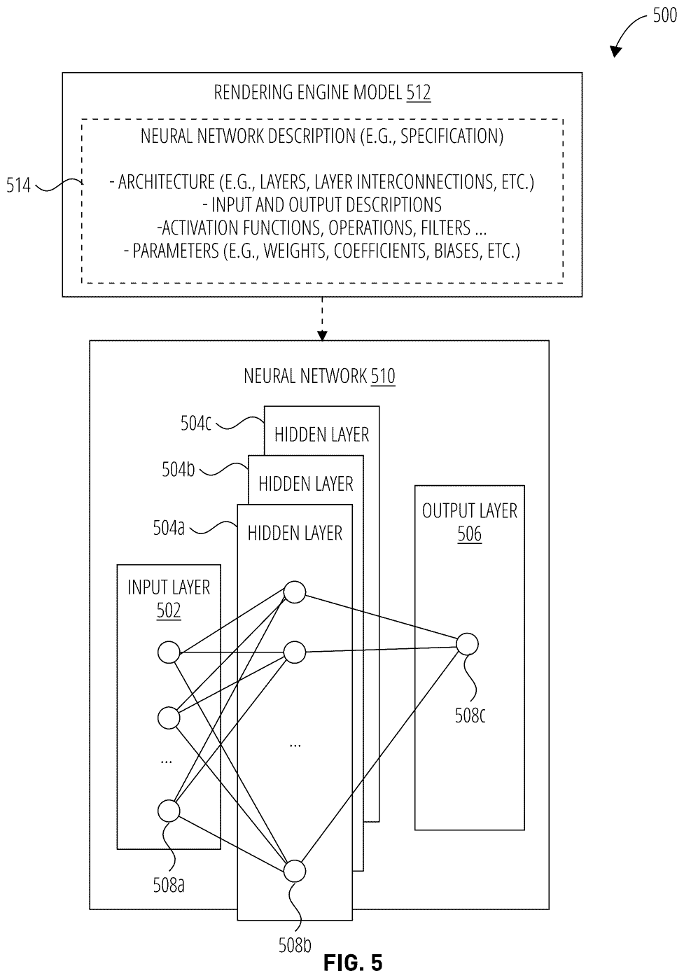

FIG. 5 illustrates an example neural network architecture.

Architecture 500 includes a neural network 510 defined by an example neural network description 514 in rendering engine model (neural controller) 512. The neural network 510 can represent a neural network implementation of a rendering engine for rendering media data. The neural network description 514 can include a full specification of the neural network 510, including the neural network architecture 500. For example, the neural network description 514 can include a description or specification of the architecture 500 of the neural network 510 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

The neural network 510 reflects the architecture 500 defined in the input layer 502. In this example, the neural network 510 includes an input layer 502, which includes input data, such as historical data and sensor readings related to the algae reactor, such as temperature, pH, nutrient levels, and CO₂ concentration. In one illustrative example, the input layer 502 can include data representing a portion of the input media data such as a patch of data (e.g., temperature, pH, nutrient levels, and CO₂ concentration) corresponding to the input data (e.g., historical data and sensor readings related to the algae reactor). The neural network 510 includes hidden layers 504a through 504n (collectively “504” hereinafter). The hidden layers 504 can include n number of hidden layers, where n is an integer greater than or equal to one. The number of hidden layers can include as many layers as needed for a desired processing outcome and/or rendering intent.

The neural network 510 further includes an output layer 506 that provides an output (e.g., rendering output) resulting from the processing performed by the hidden layers 504. In one illustrative example, the output layer 506 can provide predicted CO₂ consumption rates. The neural network 510 in this example is a multi-layer neural network of interconnected nodes. Each node can represent a piece of information. Information associated with the nodes is shared among the different layers and each layer retains information as information is processed. In some cases, the neural network 510 can include a feed-forward neural network, in which case there are no feedback connections where outputs of the neural network are fed back into itself. In other cases, the neural network 510 can include a recurrent neural network, which can have loops that allow information to be carried across nodes while reading in input. Information can be exchanged between nodes through node-to-node interconnections between the various layers.

Nodes of the input layer 502 can activate a set of nodes in the first hidden layer 504a. For example, as shown, each of the input nodes of the input layer 502 is connected to each of the nodes of the first hidden layer 504a. The nodes of the hidden layers hidden layer 504a can transform the information of each input node by applying activation functions to the information. The information derived from the transformation can then be passed to and can activate the nodes of the next hidden layer (e.g., 504b), which can perform their own designated functions. Example functions include convolutional, up-sampling, data transformation, pooling, and/or any other suitable functions. The output of the hidden layer (e.g., 504b) can then activate nodes of the next hidden layer (e.g., 504n), and so on. The output of the last hidden layer can activate one or more nodes of the output layer 506, at which point an output is provided. In some cases, while nodes (e.g., nodes 508a, 508b, 508c) in the neural network 510 are shown as having multiple output lines, a node has a single output and all lines shown as being output from a node represent the same output value. In some cases, each node or interconnection between nodes can have a weight that is a set of parameters derived from training the neural network 510.

For example, an interconnection between nodes can represent a piece of information learned about the interconnected nodes. The interconnection can have a numeric weight that can be tuned (e.g., based on a training dataset), allowing the neural network 510 to be adaptive to inputs and able to learn as more data is processed. The neural network 510 can be pre-trained to process the features from the data in the input layer 502 using the different hidden layers 504 in order to provide the output through the output layer 506. In an example in which the neural network 510 is used to identify predicted CO₂ consumption rates, the neural network 510 can be trained using training data that includes relevant features from historical data and sensor readings related to the algae reactor, such as temperature, pH, nutrient levels, and CO₂ concentration. For instance, training data can be input into the neural network 510, which can be processed by the neural network 510 to generate outputs which can be used to tune one or more aspects of the neural network 510, such as weights, biases, etc. In some cases, the neural network 510 can adjust weights of nodes using a training process called backpropagation. Backpropagation can include a forward pass, a loss function, a backward pass, and a weight update. The forward pass, loss function, backward pass, and parameter update is performed for one training iteration.

The process can be repeated for a certain number of iterations for each set of training media data until the weights of the layers are accurately tuned. For a first training iteration for the neural network 510, the output can include values that do not give preference to any particular class due to the weights being randomly selected at initialization. For example, if the output is a vector with probabilities that the object includes different product(s) and/or different users, the probability value for predicted CO₂ consumption rates may be equal or at least very similar. With the initial weights, the neural network 510 is unable to determine low level features and thus cannot make an accurate determination of what the classification of the object might be. A loss function can be used to analyze errors in the output. Any suitable loss function definition can be used. The loss (or error) can be high for the first training dataset (e.g., relevant features from historical data and sensor readings related to the algae reactor, such as temperature, pH, nutrient levels, and CO₂ concentration) since the actual values will be different than the predicted output.

The goal of training is to minimize the amount of loss so that the predicted output comports with a target or ideal output. The neural network 510 can perform a backward pass by determining which inputs (weights) most contributed to the loss of the neural network 510, and can adjust the weights so that the loss decreases and is eventually minimized. A derivative of the loss with respect to the weights can be computed to determine the weights that contributed most to the loss of the neural network 510. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 510 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for down-sampling), and fully connected layers. In other examples, the neural network 510 can represent any other neural or deep learning network, such as an autoencoder, a deep belief nets (DBNs), a recurrent neural networks (RNNs), etc.

The functions performed in the processes and methods may be implemented in differing orders. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. A method for tracking carbon capture within algae reactors, the method comprising:
monitoring an algal slurry that is processed into a dried algal biomass within an algae reactor;
measuring one or more components of the dried algal biomass;
querying a database for at least one factor of a mass fraction, a conversion factor, or a formula for a composition of one or more species of algae in the dried algal biomass;
identifying a net mass of the dried algal biomass based on a mass of one or more of the components including at least water;
determining an amount of carbon dioxide (CO₂) that has been consumed based on the identified net mass and the at least one queried factor; and
modifying one or more operational parameters of the algae reactor based the determined amount of CO₂, wherein the modified operational parameters changes one or more conditions within the algae reactor.

2. The method of claim 1, further comprising processing the algal slurry into the dried algal biomass by:
processing the algal slurry into a dewatered algal slurry; and
processing the dewatered algal slurry into the dried algal biomass.

3. The method of claim 2, wherein processing the algal slurry into the dewatered algal slurry is based on at least one of a biomass membrane separator, filtration, centrifugation, or flocculation and includes determining that the dewatered algal slurry has a first water content below a target dewatering threshold.

4. The method of claim 3, wherein the biomass membrane separator includes a semi-permeable membrane having one or more pores of a specific size to allow passage of one or more of liquids, dissolved substances, and molecules smaller than the specific size while retaining biomass and algae cells larger than the specific size.

5. The method of claim 2, wherein processing the dewatered algal slurry into the dried algal biomass includes:
utilizing thermal energy to evaporate remaining water from the dewatered algal slurry; and
determining that the dried algal biomass has a second water content below a target drying threshold.

6. The method of claim 1, wherein measuring the components of the dried algal biomass includes measuring one or more of a mass of the dried algal biomass, water content of the dried algal biomass, and percentage component of one or more other components in the dried algal biomass.

7. The method of claim 6, wherein measuring the water content or the percentage component of the other components is based on mass spectrometry.

8. The method of claim 1, further comprising updating the database based on the determined amount of CO₂ and identifying an updated conversion factor for use in one or more future analyses of the algae reactor in accordance with the updated database.

9. The method of claim 1, wherein the components include at least one of biological components, bacteria, nitrogen, potassium, trace elements, minerals, or metals.

10. The method of claim 1, further comprising training a machine-learning model to predict CO₂ consumption associated with the conditions based on the determined amount of CO₂.

11. The method of claim 10, wherein training the machine-learning model includes:
extracting one or more features from historical data and sensor readings related to the algae reactor, the extracted features including one or more of temperature, pH, nutrient levels, and CO₂ concentration; and
training the machine-learning model using the extracted features with a loss function that optimizes parameters of the model to minimize errors between predicted CO₂ consumption rates and actual measured values indicative of CO₂ consumption.

12. The method of claim 11, wherein modifying the operational parameters of the algae reactor further based on the trained machine-learning model.

13. The method of claim 1, further comprising generating a report to send to recipient device over a communication network, the report including a status update indicating one or more of actions taken, an overall operational state of the algae reactor, performance, operating history, learned correlations, and recommendations.

14. A photobioreactor control system comprising:
a gas source interfacing a CO₂ concentrator; and
one or more sensors that:
monitor an algal slurry that is processed into a dried algal biomass within an algae reactor, and
measure one or more components of the dried algal biomass;
a communication interface that communicates over a communication network to query a database for at least one factor of a mass fraction, a conversion factor, or a formula for a composition of one or more species of algae in the dried algal biomass; and
a processor that executes instructions stored in memory, wherein the processor executes the instructions to:
identify a net mass of the dried algal biomass based on a mass of one or more of the components including at least water;
determine an amount of carbon dioxide (CO₂) that has been consumed based on the identified net mass and the at least one queried factor; and
modify one or more operational parameters of the algae reactor based the determined amount of CO₂, wherein the modified operational parameters changes one or more conditions within the algae reactor.

15. The photobioreactor control system of claim 14, further comprising the algae reactor that processes the algal slurry into the dried algal biomass by:
processing the algal slurry into a dewatered algal slurry; and
processing the dewatered algal slurry into the dried algal biomass.

16. The photobioreactor control system of claim 15, wherein the algae reactor processes the dried algal slurry into the dewatered algal slurry based on at least one of a biomass membrane separator, filtration, centrifugation, or flocculation, and wherein the processor executes further instructions to determine that the dewatered algal slurry has a first water content below a target dewatering threshold.

17. The photobioreactor control system of claim 14, wherein the sensors measure the components of the dried algal biomass by measuring one or more of a mass of the dried algal biomass, water content of the dried algal biomass, and percentage component of one or more other components in the dried algal biomass.

18. The photobioreactor control system of claim 17, wherein the sensors associated with a mass spectrometer, and wherein the mass spectrometer measures the water content or the percentage component of the other components.

19. The photobioreactor control system of claim 14, further comprising a machine learning model, and wherein the processor executes further instructions to train the machine-learning model to predict CO₂ consumption associated with the conditions based on the determined amount of CO₂.

20. The photobioreactor control system of claim 19, wherein the processor trains the machine-learning model by:
extracting one or more features from historical data and sensor readings related to the algae reactor, the extracted features including one or more of temperature, pH, nutrient levels, and CO₂ concentration; and
training the machine-learning model using the extracted features with a loss function that optimizes parameters of the model to minimize errors between predicted CO₂ consumption rates and actual measured values indicative of CO₂ consumption.

21. A non-transitory computer-readable storage medium, the computer-readable storage medium including instructions executable by a computer to perform a method for tracking carbon capture within algae reactors, the method comprising:
monitoring an algal slurry that is processed into a dried algal biomass within an algae reactor;
measuring one or more components of the dried algal biomass;
querying a database for at least one factor of a mass fraction, a conversion factor, or a formula for a composition of one or more species of algae in the dried algal biomass;
identifying a net mass of the dried algal biomass based on a mass of one or more of the components including at least water;
determine an amount of carbon dioxide (CO₂) that has been consumed based on the identified net mass and the at least one queried factor; and
modifying one or more operational parameters of the algae reactor based the determined amount of CO₂, wherein the modified operational parameters change one or more conditions within the algae reactor.
